(19) United States
(12) Patent Application Publication
(10) Pub. No.: US 2002/0128313 A1
(43) Pub. Date: Sep. 12, 2002

(54) Method for improved pregnancy management and related composition

(76) Inventors: Eric J. Schmitt, Villaines-la-Juhel (FR); James R. Bradford, Kalamazoo, MI (US)

Correspondence Address: Andrew M. Solomon, Pharmacia & Upjohn Company, Global Intellectual Property, 301 Henrietta Street, Kalamazoo, MI, 49001, US

(21) Appl. No.: 09/898,975

(22) Filed: Jul. 3, 2001

Related U.S. Application Data
(60) Provisional application No. 60/217,054, filed on Jul. 10, 2000.

(51) Int. Cl. A61K 31/5575

(52) U.S. Cl. 514/530; 800/21; 514/559

(57) ABSTRACT

A method for reducing the average number of insemination administrations per herd comprising the step of administering to the herd native or extended semen with a simultaneous or sequential administration of prostaglandin is provided. Also provided is a novel composition of matter which includes either native semen, extended semen or a semen extender, one or more prostaglandins and one or more antibioitics.

### CROSS-REFERENCE TO RELATED APPLICATION

This application claims the benefit of the following provisional application: U.S. Ser. No. 60/217,054, filed Jul. 10, 2000, under 35 USC 119(e)(i).

### BACKGROUND OF THE INVENTION

1. Field of the Invention

This invention pertains to the use of luteolytic agents, especially prostaglandins, and especially Prostaglandin F_{2α}, and its analogs in native or extended boar semen to reduce the number of artificial inseminations required to obtain normal or increased pregnancy rates in sows. The composition of semen may also include one or more antibiotics.

2. Technology Description

Because of their luteolytic effects, the prostaglandin family, and in particular the natural and synthetic analogs of Prostaglandin F_{2α}(PGF_{2α}), are widely used as an aid in the artificial insemination practice common in the reproduction of food animals. One report describes stimulation of libido in pubertal and mature boars with PGF₂,, analogs (Szurop et al., 1985). Several reports indicate that the addition of 5 mg of PGF_{2α}in extended boar sperm resulted in an increased number of piglets born alive and increased the likelihood of a sow to become pregnant (Gil et al., 1998). Similarly, administration of PGF_{2α}, helped alleviate the effect of seasonal infertility in sows by maintaining the number of piglets born per litter and the number of sows farrowing at a constant level year round (Pena et al., 1998). Takacs et al. (1985) have discussed the effect of PGF_{2α}treatment of boar semen and sows on the fertility level in an artificial insemination system. Niwa et al. (1982) have discussed influences of addition of PGF_{2α}to boar semen diluent upon viability of sperm, conception rate and subsequent number of piglets born.

In the swine industry, artificial insemination is commonly used to impregnate sows. In the normal practice, semen is collected from boars and analyzed for sperm cells concentration, total sperm cells number and the viability of the sperm cells. Subsequently, the semen is commonly diluted with an extender to provide a semen composition suitable for artificial insemination. The semen extenders typically contain nutrients to preserve the viability of the sperm cells, and, optionally, antibiotics to preserve the composition from bacterial degradation. Typically, the extenders are milk, egg yolk, egg yolk-glucose or milk-glucose based.

In practice,the length of estrus in the porcine can last from 36 hours to 100 hours. As a consequencethe females are inseminated at several times during each estrus period in order to increase the probability of the insemination resulting in pregnancy. However, each insemination administration requires time and costs in terms of personnel, materials and the like. The number of inseminations required to achieve a 85% to 90% conception rate can range from 2 to 4 administrations, with 3 being typical. In general, the insemination process requires from about ten to twenty minutes. In large herds, a reduction in the total number number of inseminations, as averaged over the herd, can be seen to provide substantial savings in labor, semen, semen extender and number of sperm donor boars. Further, the reduced number of inseminations reduces animal handling and thereby increases the welfare of the animals.

In addition, the use of antibiotics in boar semen or extended boar semen is known. Quite often the antibiotic is added for the purpose of improved storage conditions and in other instances to reduce possible defects in the litter. See, for example Cerovsky, 1986, Hovorka (1985) and Underwood (1982). Many semen extenders already contain one or a combination of antibiotics as a basis for their composition. . Legal requirements may exist for antibiotic treatment of extended semen for biosecurity reasons when semen must travel within or outside of a country as exemplified in the The European Union Council Directive of Jun. 26, 1990 (90/429/EEC). This legislation recommends that an effective combination of antibiotics, in particular against leptospires and mycoplasmas, must be added to the semen after final dilution. This combination must produce an effect at least equivalent to the following dilutions:

Not less than: 500 IU per mL Streptomycin

500 IU pre mL Penicillin

150 pg per mL Lincomycin

300 pg per mL Spectinomycin.

Despite the above teachings, there still exists a need in the art for a method for reducing the number of insemination administrations per sow herd. In addition, there exists a need for a composition that can provide the dual benefit of having both prostaglandin and antibiotic.

### BRIEF SUMMARY OF THE INVENTION

In accordance with the present invention a novel method for reducing the average number of insemination administrations per sow herd is provided. More specifically the method comprises administering to the herd native or extended semen with a simultaneous or sequential administration of prostaglandin.

In preferred embodiments the prostaglandin is PGF_{2α}(and any natural or synthetic compound of the PGF_{2α}family of prostaglandins) and the method is capable in reducing the number of administrations required to achieve a greater than 80% pregnancy rate in a herd by between about 20 and about 80% as compared to methods where PGF_{2α}is not administered. In particularly preferred embodiments, the method is used for the treatment of pigs (sows).

In still another embodiment a novel composition of matter is provided. The composition comprises native semen, extended semen or a semen extender, one or more prostaglandins and one or more antibiotics. In preferred embodiments the prostaglandin is PGF_{2α}and the antibiotic is either ceftiofur or a combination of LINCOMYCIN® and SPECTINOMYCIN®.

An object of the present invention is to provide a method for reducing the number of inseminations per herd.

Still another object of the present invention is to provide a novel composition to be used in artificial insemination.

These, and other objects, will readily be apparent to those skilled in the art as reference is made to the detailed description of the preferred embodiment.

### DETAILED DESCRIPTION OF THE PREFERRED EMBODIMENT

In describing the preferred embodiment, certain terminology will be utilized for the sake of clarity. Such terminology is intended to encompass the recited embodiment, as well as all technical equivalents which operate in a similar manner for a similar purpose to achieve a similar result.

The present invention is directed to a method for reducing the number of insemination administrations per herd in order to achieve a desired pregnancy rate of at least 80% in the herd by the administration of a prostaglandin with the native or extended semen. In preferred embodiments the target pregnancy rate is between 80 - 100% with a rate of 80-90% being most common. Of course a 100% pregnancy rate is ideal but is not always practically possible.

In practice, the invention is particularly geared to the insemination of sows, but the insemination of other animals including, but not limited to cows, sheep, horses, goats, deer and the like are expressly contemplated.

Further the number of inseminations per herd is to be reduced by between about 20% and about 80% as compared to insemination methods not including the prostaglandin. This is accomplished by reducing what would be 2, 3, 4 or 5 inseminations to 1, 2, 3 or 4 inseminations. The greatest reduction in the number of inseminations is by going from 5 to 1 while the least reduction is by going from 5 to 4.

The prostaglandin to be used should be one known in the art to enhance pregnancy. In practice the preferred prostaglandin is PGF_{2α}, including analogs and synthetic analogs. A commercial product particularly useful in the present invention is sold under the name LUTALYSE® Sterile Solution by Upjohn Animal Health. One milliliter of LUTALYSE® Sterile Solution contains 5 mg of active ingredient, PGF_{2α}, (dinoprost as the tromethamine salt).

The prostaglandin may be directly added to the native semen or semen extender prior to insemination or may be separately administered just after or just before administration of the native or dilute semen. Any novel semen extender composition containing any amount of prostaglandin to obtain a final concentration of 1 to 10 mg of active prostaglandin per insemination dose is also contemplated. In practice the amount of prostaglandin added to aid in the process ranges from between about 1 to about 10 mg of active per sow, more preferably between about 1.5 to about 5 mg per sow and most preferably about 2.5 mg per sow.

The semen used for insemination may either be in its native or extended form. In practice because of farm economics it is preferable to dilute the native semen using a commercially acceptable extender. In practice for every one part of native semen, about 2 to about 10 parts of extender are used, with a ratio of 1 part native semen to about 3 to about 7 parts extender being more preferred and a ratio of 1 part native semen to about 4 parts extender being most preferred.

The amount of semen used to inseminate the animal typically ranges from between about 5 to about 250 ml of native semen, more preferably between about 10 and about 50 ml of native semen. To the extent that the semen is extended, the amount of extended semen is simply the multiple of the extension. For example, if the sow is inseminated with 80 ml of extended semen where the extension is 4 parts extender to 1 part native semen, the amount of native semen used in the insemination is 16 ml.

When extended, the extender is made of either milk, egg yolk, egg yolk-glucose or milk-glucose. Mixtures of the above are also considered as falling within the scope of the invention. Examples of commercially available extenders are sold under the names of Kiev, Modified Modena® or Beltsville Thaw Solution (BTS); these commonly available extenders allow for conservation of the semen for up to 3 days. Long term extenders, allowing conservation of semen for up to 5 or 7 days are Androhep®), MR-A®, X-Cell™, Zorpva, and Reading.

Also optionally present in the native semen, extended semen or extender is one or more antibiotics. Those commonly used for animal health treatment are considered as specifically falling within the scope of the invention. The antibiotics can be selected from the following classes of antibiotics: aminoglycosides, amphenicols, beta-lactams, macrolides, lincosamides, NOVOBIOCIN, SPECTINOMYCIN®, sulfa drugs and tetracyclines and mixtures thereof. Examples of such antibiotics include, but are not limited to the ceftiofur family of cephalosporins, including the sodium salt, hydrochloride salt and free acid, Spectinomycin, Lincomycin, and mixtures thereof. Examples of commercially useful antibiotics include NAXCEL®, EXCENEL® and LINCO-SPECTIN®, all sold by Upjohn Animal Health. The antibiotic is present in either the native or extended semen or in the extender such that the dosage which is to be administered to the patient ranges from about 1 to about 10 mg of the active antibioitic ingredient per dosage form/kg body weight of patient, more preferably about 2 to 6 mg/kg and most preferably between about 3 to 5 mg/kg.

The invention is described in greater detail by the following non-limiting examples.

### EXAMPLE 1

Females gilts were assigned to either a treatment group or a control group at breeding based on ear tag number (even numbered females to the treated group, odd numbered to the control group). Females in the control group were inseminated with extended boar semen and 0.5 ml of LUTALYSE® Sterile Solution added at the time of insemination. The LUTALYSE® was injected into the breeding catheter, the catheter was inserted into the cervix and the catheter was then flushed with 80 mnL of extended semen. Females in the control group were inseminated with extended boar semen. Pig Champ records were used as the recording method and the records were analyzed for farrowing rate (FR), adjusted farrowing rate, and litter size. Records with incomplete or conflicting data were excluded from the analysis.

Records from 2274 animals were available for analysis (1575 gilts and 699 parity 1). Gilts inseminated with LUTALYSE(® treated semen had higher farrowing rates and lower service periods per pregnancy than gilts inseminated with untreated semen. A service period is the duration of one estrus. There was no difference in total born or born live between the two groups. In parity 1 females, the farrowing rate at subsequent farrowing was higher in the treated group. There was no difference in service periods per pregnancy (SP/P), total born (TB) and born alive. Across both parities, there was a significant difference in farrowing rate and service periods per pregnancy in favor of the LUTALYSE® treated group. See Tables 1-4.

TABLE 1
Gilts Farrowing rate, service period/pregnancy and total born
by treatment

| | LUTALYSE ® | Control | P value |
|---|---|---|---|
| N | 769 | 806 | |
| FR | 84.9 | 81.5 | <.02 |
| SP/P | 1.26 | 1.38 | <.03 |
| TB | 10.1 | 10.1 | <.17 |

TABLE 2
Parity 1 Farrowing rate, service period/pregnancy and total born by
treatment

| | LUTALYSE ® | Control | P value |
|---|---|---|---|
| N | 373 | 326 | |
| FR | 85.8 | 81.4 | <.05 |
| SP/P | 1.29 | 1.31 | <.06 |
| TB | 10.2 | 10.1 | <.27 |

TABLE 3
Gilts and parity 1 Farrowing rate, service period/pregnancy and total
born by treatment.

| | LUTALYSE ® | Control | P value |
|---|---|---|---|
| N | 1142 | 1132 | |
| FR | 85.2 | 81.5 | <.03 |
| SP/P | 1.27 | 1.36 | <.03 |
| TB | 10.1 | 10.1 | <.17 |

In both the hot season (<8/31) and the moderate season (>8/31), the treated gilts outperformed the controls with respect to FR and SP/P.

TABLE 4
Farrowing rate by season bred and treatment group

| Breed | | | |
|---|---|---|---|
| date | LUTALYSE ® | Control | P value |
| 6/01- | 86.8 | 82.0 | <.03 |
| 8/31 | | | |
| >8/31 | 82.9 | 80.1 | <.03 |

P(seasonal effect) <.03

TABLE 5
Service periods per pregnancy by season bred and treatment group

| Breed | | | |
|---|---|---|---|
| date | LUTALYSE ® | Control | P value |
| 6/01- | 1.17 | 1.31 | <.04 |
| 8/31 | | | |
| >8/31 | 1.35 | 1.47 | <.05 |

P(seasonal effect) <.05

In this trial, gilts responded positively to the addition of LUTALYSE® to extended boar semen. Farrowing rate and service periods per pregnancy were positively affected. Litter size was unaffected. Parity 1 females responded to LUTALYSE® addition to extended semen with a higher farrowing rate. In gilts, the effect of adding LUTALYSE® was significantly greater in the hot season than in the moderate season and significantly better in both seasons than controls.

### EXAMPLE 2

A semen extender is prepared by mixing together the following components:

| | | |
|---|---|---|
| Egg yolk | 300 | ml |
| Distilled water | 680 | ml |
| Glucose | 30 | g |
| An antibiotic chosen from | | |
| Ceftiofur | 1.0 | g |
| AND/OR | | |
| LINCO-SPECTIN ® | 20 | ml |
| Prostaglandin F2α (as LUTALYSE ® Sterile Solution) | 62.5 | mg (12.5 ml |
| LUTALYSE ®) | | |

4 parts of the above mixture is used to dilute 1 part of native boar semen to yield composition which is to be used to artificially inseminate sows.

Having described the invention in detail and by reference to the preferred embodiments thereof, it will be apparent that modifications and variations are possible without departing from the scope of the appended claims.

What is claimed is:

1. A method for reducing the average number of insemination administrations per herd comprising the step of administering to the herd native or extended semen with a simultaneous or sequential administration of prostaglandin.

2. The method according to claim 1 wherein said herd is a herd of sows, cows, horses, sheep, goats or deer.

3. The method according to claim 2 wherein said herd is a herd of sows.

4. The method according to claim 1 wherein the number of administrations to achieve a herd pregnancy rate of at least 80% is reduced by 20 to 80% as compared to the number of administrations required where no prostaglandin is administered.

5. The method according to claim 1 wherein the prostaglandin is PGF_{2α}.

6. The method according to claim 1 wherein said prostaglandin is simultaneously or sequentially added with the semen required for insemination.

7. The method according to claim 6 wherein the prostaglandin is included with native semen, extended semen or with a semen extender.

8. The method according to claim 1 comprising the additional step of administering an antibiotic to the members of the herd.

9. A composition of matter comprising:
(a) native semen, extended semen or a semen extender;
(b) one or more prostaglandins: and
(c) one or more antibiotics.

10. The composition according to claim 9 wherein (b) is prostaglandin F₂, and (c) is either ceftiofur, LINCOMYCIN, SPECTINOMYCIN or mixtures thereof.

11. The composition according to claim 10 wherein (a) is a semen extender.
